(19) United States
(12) Patent Application Publication
(10) Pub. No.: US 2017/0281772 A1
(43) Pub. Date: Oct. 5, 2017

(54) A NEURAL SUBSTRATE FOR SUGAR PREFERENCE

(71) Applicants: Charles Zuker, New York, NY (US); Ryan T. Lessard, New York, NY (US); Yossef Goffer, Brooklyn, NY (US)

(72) Inventors: Charles Zuker, New York, NY (US); Ryan T. Lessard, New York, NY (US); Yossef Goffer, Brooklyn, NY (US)

(73) Assignee: The Trustees of Columbia University in the City of New York, New York, NY (US)

(21) Appl. No.: 15/512,093

(22) PCT Filed: Sep. 18, 2015
(86) PCT No.: PCT/US15/50999

§ 371 Date: Mar. 17, 2017

Related U.S. Application Data
(60) Provisional application No. 62/159,060, filed on May 8, 2015.
(60) Provisional application No. 62/052,259, filed on Sep. 18, 2014.

(51) Int. Cl. A61K 45/06; A61K 31/70; A61K 35/30; A61K 31/198

(52) U.S. Cl. CPC A61K 45/06; A61K 31/198; A61K 31/70; A61K 35/30; A61K 31/00

(57) ABSTRACT

This invention concerns a composition and a method of modulating the craving and/or desire for natural sugar in a subject comprising: agonizing or stimulating or antagonizing or silencing a selective group of neurons in the cadual nucleus of the solitary tract (cNST) of the brain in the subject, whether directly or via the gut or gut-brain axis.

This application claims the priority of U.S. Provisional Application No. 62/159,060, filed May 8, 2015, and claims priority of U.S. Provisional Application No. 62/052,259, filed Sep. 18, 2014, the contents of each of which are hereby incorporated by reference.

All publications and other references mentioned herein are incorporated by reference in their entirety, as if each individual publication or reference were specifically and individually indicated to be incorporated by reference. Publications and references cited herein are not admitted to be prior art.

Throughout this application, various publications are referenced, including referenced in parenthesis. Full citations for publications referenced in parenthesis may be found listed at the end of the specification immediately preceding the claims. The disclosures of all referenced publications in their entireties are hereby incorporated by reference into this application in order to more fully describe the state of the art to which this invention pertains.

### BACKGROUND OF THE INVENTION

Diabetes and obesity have reached epidemic levels worldwide, affecting over 300 and 500 million people respectively. The World Health Organization predicts that diabetes will become the 7^{th} leading cause of death by 2030 without novel treatment modalities. The excessive consumption of sugar is thought to contribute significantly to both of these diseases. Questions remain regarding why animals are intensely attracted to sugar.

Artificial sweeteners do not stimulate or trigger a “sugar preference” behavior, and may in fact stimulate sugar craving (Yang) . The consumer industry, particularly the sweetened beverage industry, is facing a major challenge in trying to reduce sugar levels from their primary products (non-diet drinks) while maintaining their attractive flavor profile AND, most importantly, their sugar “appetitiveness”.

### SUMMARY OF THE INVENTION

This invention concerns a method of modulating the craving and/or desire for natural sugar in a subject, comprising agonizing or stimulating or antagonizing or silencing a selective group of neurons in the caudal nucleus of the solitary tract (cNST) of the brain in the subject, either directly or via the gut or gut-brain axis.

This invention also concerns a composition for modulating the craving and/or desire for natural sugar, wherein the composition agonizes or stimulates or antagonizes or silences a selective group of neurons in the caudal nucleus of the solitary tract (cNST) of the brain in the subject, either directly or via the gut or gut-brain axis.

This invention also concerns a method for identifying a composition or agent for modulating the craving or desire for natural sugar comprising:
a) administering a natural sugar to a mouse;
b) detecting neural activity in the neurons in the caudal nucleus of the solitary tract (cNST) of the brain;
c) administering the composition, or agent to the mouse;
d) detecting neural activity in the neurons in the caudal nucleus of the solitary tract (cNST) of the brain;
e) comparing the neural activity in step (d) with the neural activity in step (b),
wherein a decrease or less neural activity in step (d) as compared to step (b) indicates that the agent or composition is decreasing the craving or desire for natural sugar, and an increase or more neural activity in step (d) as compared to step (b) indicates the agent or composition is increasing the craving or desire for natural sugar.

This invention also concerns a method of increasing an individual's preference for a consumer product, or maintaining an individual's preference for a consumer product while reducing its metabolizable, sugar content, which comprises adding to said consumer product a non-metabolizable, sugar analog capable of activating a gut-brain sweet preference circuit in an amount effective to activate such circuit.

### BRIEF DESCRIPTION OF THE DRAWINGS

FIG. 1

Results of experiment demonstrating that animals change preference between artificial sweetener and sucrose after one exposure to sucrose.

FIG. 2

Results of experiment demonstrating that animals lacking the ability to taste sweet develop a preference for sucrose.

FIG. 3

cFos staining in the caudal nucleus of the solitary tract. Animals challenged with water (FIG. 3A), sucrose (FIG. 3B), or artificial sweetener (FIG. 3C). Tasteless (TrpM5 knockout) animals were given water (FIG. 3D) or sucrose (FIG. 3E). Green: c-fos antibody staining. Magenta: a neural stain (neurotrace). Neurons in animals given sucrose are robustly labeled while animals given water or sweetener show almost no labeled cells at all. An identical pattern of labeling is visible in both wild type and tasteless mice, indicating that the labeling is taste independent.

FIG. 4A-4C

cFos staining in the nucleus of the solitary tract after direct infusion to the gut. Wild type animals infused directly into the gut with water (FIG. 4A), sucrose (FIG. 4B), or artificial sweetener (FIG. 4C) . Green: c-fos antibody staining. Magenta: a neural stain (neurotrace). Neurons in animals given sucrose are robustly labeled while animals given water or sweetener show almost no labeled cells at all.

FIG. 5

Results of experiment demonstrating that silencing of neural activity in the cNST during sucrose exposure blocks animals from preferring sugar. After the silencing drug is washed away, animals do indeed develop the usual sugar preference.

FIG. 6

Results of experiment demonstrating that activation of sugar-responsive neurons in the cNST is attractive.

FIG. 7

Results of experiment demonstrating that animals strongly prefer water coupled with light activation of channelrhodopsin expressing neurons to water alone.

FIG. 8

Activation of sugar-responsive neurons in the NST forms a preference to a neutral cue. The Preference Index indicates whether the animals preferred a low concentration sodium chloride solution (positive preference index) or preferred water (negative preference index).

FIG. 9

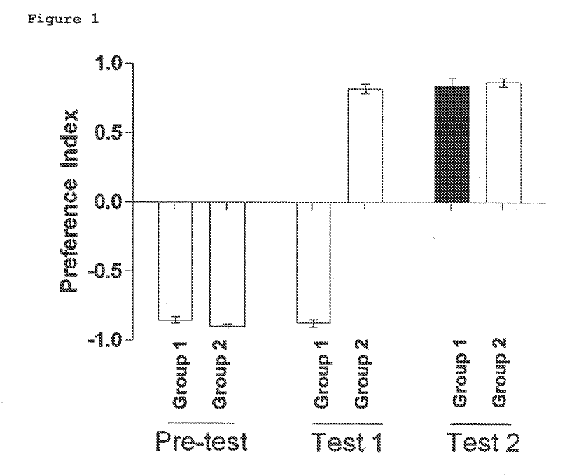

Results of experiment demonstrating that the presence of MDG transforms a sucralose solution into the preferred Artificial Sweetener.

### DETAILED DESCRIPTION OF THE INVENTION

### General Techniques and Definitions

Unless specifically defined otherwise, all technical and scientific terms and techniques used herein shall be taken, to have the same meaning as commonly understood by one of ordinary skill in the art.

As used herein, “gut-brain axis” refers to signaling taking place between the gastrointestinal tract and the nervous system. For example, bioactive molecules., nutrients and metabolites in the GI tract can activate gastrointestinal cells (for example entero endocrine cells) and signal directly or indirectly through the vagal nerve to brain circuits involved in metabolism, physiology, immunity, motivation and behavior.

As used herein, the term “sugar analog” means a chemical compound that is structurally similar to a naturally occurring sugar, but differs in respect to one or more structural atoms. For example, one atom within a sugar may be replaced with a different atom or one functional group of a sugar may be replaced by a different functional group. For example, a carbon may be replaced.

As used herein, the term “non-metabolizable” means a compound which is not metabolized under normal physiological conditions within the body of an individual to whom the compound is administered.

Any non-metabolizable sugar analog can be readily tested to determine whether it is capable of activating a gut-brain sweet preference circuit using the techniques described in this application.

### EMBODIMENTS

This invention concerns a method of modulating the craving and/or desire for natural sugar in a subject, comprising agonizing or stimulating a selective group of neurons in the caudal nucleus of the solitary tract (cNST) of the brain in the subject, either directly or via the gut or gut-brain axis.

This invention concerns a method of modulating the craving and/or desire for natural sugar in a subject, comprising antagonizing or silencing a selective group of neurons in the caudal nucleus of the solitary tract (cNST) of the brain in the subject, either directly or via the gut or gut-brain axis.

In one embodiment the neurons are antagonized or silenced by the administration of a pharmaceutical composition to the subject.

In one embodiment the neurons are antagonized or silenced by the administration of a neural silencer to the subject.

In one embodiment the neural silencer is a glutamate receptor antagonist.

In one embodiment the neural silencer is NBQX.

In one embodiment the neurons are agonized or stimulated or antagonized or silenced before or during ingestion of a natural sugar or a food or beverage product containing natural sugar.

In one embodiment the pharmaceutical composition is administered before or during the ingestion of a natural sugar or a food or beverage product containing natural sugar.

In one embodiment the neural silencer is administered before or during the ingestion of a natural sugar or a food or beverage product containing natural sugar.

This invention also concerns a composition for modulating the craving and/or desire for natural sugar, wherein the composition antagonizes or silences a selective group of neurons in the caudal nucleus of the solitary tract (cNST) of the brain in the subject, either directly or via the gut or gut-brain axis. In one embodiment, the invention concerns a food or beverage product comprising such a composition.

This invention also concerns a composition for modulating the craving and/or desire for natural sugar, wherein the composition agonizes or stimulates a selective group of neurons in the caudal nucleus of the solitary tract (cNST) of the brain in the subject, either directly or via the gut or gut-brain axis. In one embodiment, the invention concerns a food or beverage product comprising such a composition.

In some embodiments the subject is a mammal.

In some embodiments the subject is a mouse.

In some embodiments the subject is a human.

This invention also concerns a method for identifying a composition or agent for modulating the craving or desire for natural sugar comprising:
a) administering a natural sugar to a mouse;
b) detecting neural activity in the neurons in the caudal nucleus of the solitary tract (cNST) of the brain;
c) administering the composition or agent to the mouse;
d) detecting neural activity in the neurons in the caudal nucleus of the solitary tract (cNST) of the brain;
e) comparing the neural activity in step (d) with the neural activity in step (b),
wherein a decrease or less activity in step (d) as compared to step (b) indicates that the agent or composition is decreasing the craving or desire for natural sugar, and an increase or more activity in step (d) as compared to step (b) indicates the agent or composition is increasing the craving or desire for natural sugar.

In one embodiment, detecting neural activity in the neurons in the caudal nucleus of the solitary tract (cNST) of the brain is accomplished by staining the brain of the mouse.

This invention also concerns a method of increasing an individual's preference for a consumer product, or maintaining an individual's preference for a consumer product while reducing its metabolizable, sugar content, which comprises adding to said consumer product a non-metabolizable sugar analog capable of activating a gut-brain sweet preference circuit in an amount effective to activate such circuit.

In one embodiment the non-metabolizable sugar analog is further-capable of activating the sweet taste receptors on the individual's tongue.

In another embodiment, the method further comprises adding to said consumer product an artificial sweetener, a sugar substitute, or a compound which activates the sweet taste receptors on the individual's tongue.

In another embodiment the non-metabolizable sugar analog is selected from Alpha-Methyl-D-Glucopyranose, Beta-D-Glucose, D-Allopyranose, Beta-L-fucose, Alpha-D-Fucose, 6-Deoxy-Alpha-D-Glucose, Beta-D-Fucose, 6-Deoxyglucose, Alpha-L-Fucose, Ribose, Alpha-L-Arabinose, Beta-L-Arabinose, Galacturonic Acid, D-Mannuronic Acid, L-Iduronic Acid, D-Glucuronic Acid, L-Glucuronic Acid, L-Glycero-D-Manno-Heptopyranose, Alpha-D-Xylopyranose, L-Xylopyranose, Beta-D-Ribopyranose 2-O-Methyl Fucose, 6-Deoxy-2-O-Methyl-Alpha-L-Galactopyranose, Methyl Alpha-D-mannoside, Methyl Alpha-galactoside, Methyl Beta-galactoside, Alpha-D-Glucose-6-Phosphate, Beta-Galactose-6-Phosphate, Alpha-D-Mannose-6-Phosphate, Beta-D-Glucose-6-Phosphate, 3,4-Epoxybutyl-Alpha-D-Glucopyranoside, 2-Deoxy-Beta-D-Galactose, 2-deoxyglucose, D-Galctopyranosyl-1-On, Gluconolactone, 1-Thio-Beta-D-Glucopyranose, O1-Pentyl-Mannose, 5 (R)-5-Fluoro-Beta-D-Xylopyranosyl-Enzyme Intermediate, D-Sorbitol, Mannitol, D-Xylitol, Beta-L-Methyl-Fucose, Alpha-L-Methyl-Fucose, Alpha-L-1-Methyl-Fucose, L-Rhamnitol, Fucitol, O3-Sulfonylgalactose, O4-Sulfonylgalactose, Gluconic Acid, Methyl(6s)-1-Thio-L-Manno-Hexodialdo-6,2-Pyranoside, 1-N-Acetyl-Beta-D-Glucosamine, Alpha-D-Glucopyranosyl-2-Carboxylic Acid Amide, D-Glucose in Linear Form, 02-Sulfo-Glucuronic Acid, 4-O-Methyl-Beta-D-Glucuronic Acid, 4-O-Methyl-Alpha-D-Glucuronic Acid, 1-Deoxy-1-Methoxycarbamido-Beta-D-Glucopyranose, Alpha-D-Galactose-1-Phosphate, D-Mannose 1-Phosphate, Alpha-D-Glucose-1-Phosphate, 1-(Isopropylthio)-Beta-Galactopyranside, 2-(Beta-D-Glucopyranosyl)-5-Methyl-1, 3, 4-Oxadiazole, 5-(3-Amino-4, 4-Dihyroxy-Butylsulfanylmethyl)-Tetrahydro-Furan-2,3,4-Triol, Beta-D-Arabinofuranose-5′-Phosphate, [(2r,3s,4s,5r)-3,4,5-Trihydroxytetrahydrofuran-2-Yl]Methyl Dihydrogen Phosphate, L-Rhamnose, Myo-Inositol, Glucarate, 3,6-Anhydro-D-Galactose-2-Sulfate, 4-Deoxy-Alpha-D-Glucose, Tetrahydrooxazine, D-Fructose-6-Phosphate, Sorbitol 6-phosphate, 2-Deoxy-Glucose-6-Phosphate, 2-Deoxy-2-Aminogalactose, Glucosamine, 2-Fluoro-2-Deoxy-Beta-D-Galactopyranose, 2-Deoxy-2-Fluoro-Alpha-D-Mannose, 2-Deoxy-2fluoro-Glucose, 2-Deoxy-2-Fluoro-Beta-D-Mannose, L-Guluronic Acid 6-Phosphate, 6-Phosphogluconic Acid, L-Myo-Inositol-1-Phosphate, 4, 6-Dideoxyglucose, 2-Deoxy-2-Fluoro-Alpha-D-Mannosyl Fluoride, 4-Deoxy-D-Glucuronic Acid, Fructose, Glucose-6-Phosphate, Beta-D-Fructopyranose, 1-Deoxy-Ribofuranose-5′-Phosphate, Tagatose, Ribose-1- Phosphate, Fructose-6- Phosphate, 5-Hydroxymethyl-Chonduritol, 3-Deoxy-D-Manno-Oct-2-Ulosonic Acid, 2-Deoxy-D-Glucitol 6-(E)-Vinylhomophosphonate, D-Treitol, Meso-Erythritol, Xylarohydroxamate, or C-(1 -Hydrogyl-Beta-D-Glucopyranosyl) Formamide.

In a preferred embodiment the non-metabolizable sugar analog is Alpha-Methyl-D-Glucopyranose.

Applicants have identified a nucleus in the brainstem that is activated by sugar, but not artificial sweetener, and is necessary to form a preference to sugar. Furthermore, applicants demonstrate that selectively activating the sugar-responsive neurons in this region of the brain is attractive and is sufficient to form a preference to a neutral stimulus. Applicants believe that these neurons are the essential substrate for the formation of sugar preference. Further, applicants believe the reason that artificial sweeteners have not been more successful in the market is due to the fact that while they taste sweet, they fail to activate this sugar preference pathway (gut-brain sweet preference circuit). The ability to manipulate these neurons may allow us to control sugar preference and treat sugar-based diseases such as obesity and diabetes. The identification of agonists and antagonists that modulate the activity of these neurons can provide important strategies for the management of eating disorders, obesity, and perhaps addictive behaviors.

In the present invention applicants demonstrate that an artificial, non-metabolizable sugar analog can be sufficient to form a “sweet preference” if it simultaneously activates the taste receptors on the tongue and the gut-brain sweet preference circuit. Applicants show this to be the case even when a sugar analog, for example MDG (Alpha-Methyl-D-Glucopyranose), is used under conditions where it is perceived as much less sweet than artificial sweeteners.

Therefore, this invention proposes that using natural or synthetic compounds that activate BOTH sweet taste receptors cells on the tongue, and the neurons mediating the gut-brain sweet brain preference circuit provide an important and novel strategy to reduce/remove sugar from consumer products (like in sugar sweetened carbonated drinks, etc.)

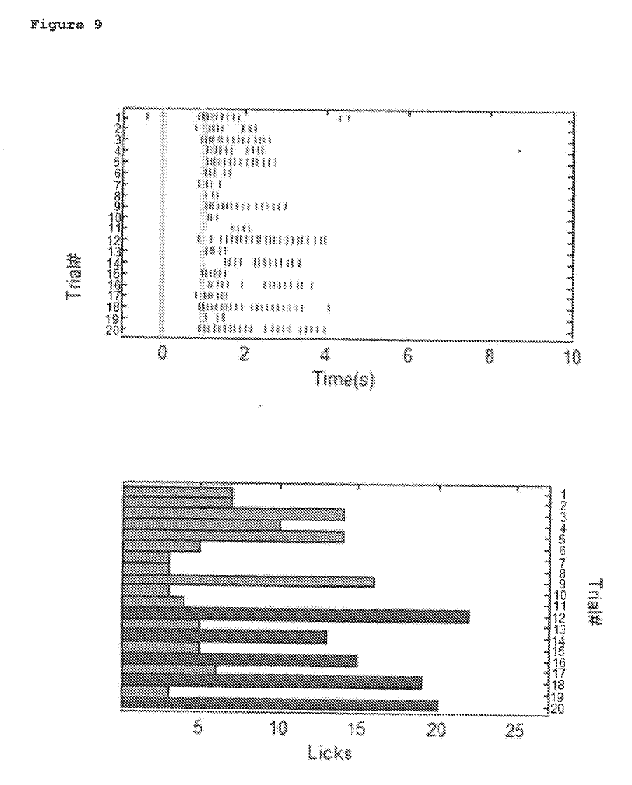

Applicants also propose that the compounds that activate the gut sweet-preference circuit need not taste sweet themselves, and instead be used in combination with artificial sweeteners, or reduced levels of natural sugar, to activate sweet receptors in the tongue and provide a mix that activated both signaling pathways (tongue and gut). See FIG. 9.

Each embodiment disclosed herein is contemplated as being applicable to each of the other disclosed embodiments. Thus, all combinations of the various elements described herein are within the scope of the invention.

This invention will be better understood by reference to the Examples which follow, but those skilled in the art will readily appreciate that the specific experiments detailed are only illustrative of the invention as defined in the claims which follow thereafter.

### EXAMPLES

Examples are provided below to facilitate a more complete understanding of the invention. The following examples illustrate some exemplary modes of practicing the invention. However, the scope of the invention is not limited to specific embodiments disclosed in these Examples, which are for purposes of illustration only.

### Example 1

### Experiment 1

To explicitly test for sugar preference in mice, applicants developed a simple behavioral assay.

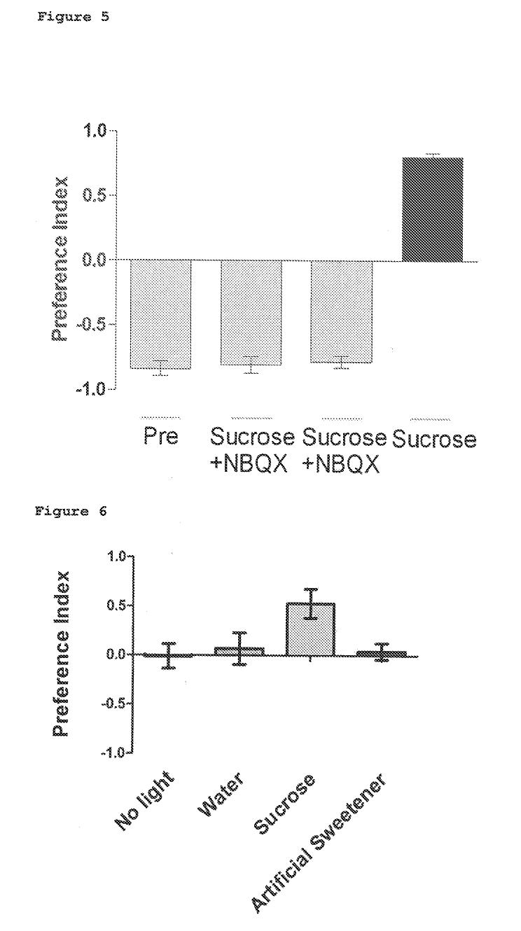

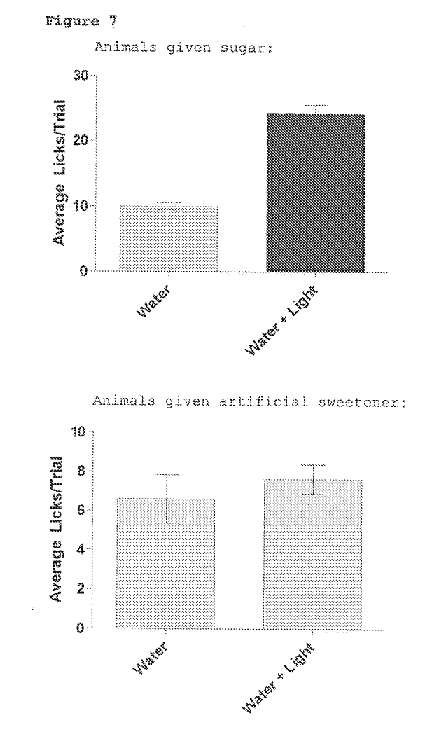

During five minute trials, two groups of five naive animals were presented with an intensely sweet, artificial compound (Acesulfame potassium or AceK) and a less sweet natural sugar (sucrose) . As expected, both groups of mice initially consumed much more AceK than sucrose. Animals were then returned to their home cage and given access to either artificial sweetener (Group 1) or natural sugar (Group 2) for one hour. Animals that received sugar in their home cage showed preference to natural sugar. Twenty four hours later, Group 1 was given natural sugar while Group 2 was given artificial sweetener. The final preference test reveals that both groups prefer the natural sugar (FIG. 5). Therefore, animals overrode their innate taste drive and formed a preference for natural sugar after a single exposure.

### Experiment 2

Applicants hypothesized that sugar preferences are formed independent of signaling in the taste pathway. Sweetness is detected by a heterodimeric G protein-coupled receptor consisting of the combination of T1R2 and T1R3 subunits. Applicants tested animals in which both receptor components are genetically lesioned for their ability to form a preference to sugar. Initially, while these animals were agnostic to the two solutions, they formed a robust preference to the sucrose solution after a single exposure.

### Experimental Details

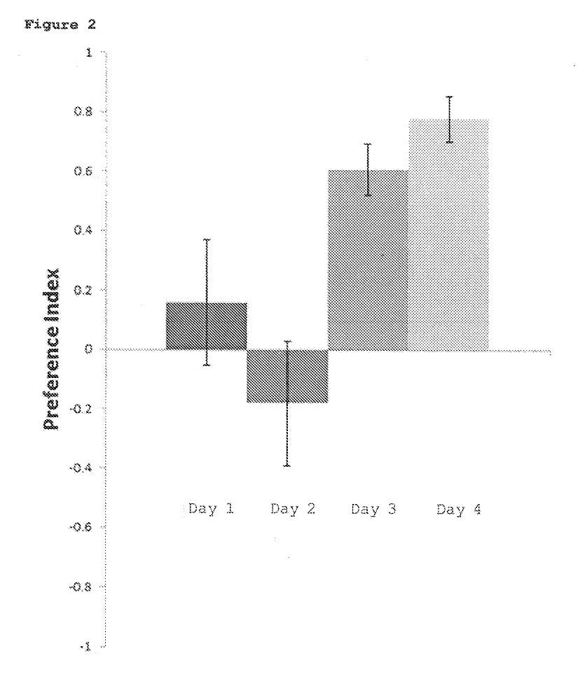

Five animals lacking the two components of the sweet taste receptor (the T1R2 and T1R3 genes) were given a choice between a low concentration of natural sugar and a high concentration of artificial sweetener. Initially, (day 1 and 2) the animals preferred neither solution. After testing on day 2, animals were then returned to their home cage and given access to the same sucrose solution. The next day, preferences for all animals were tested again. Surprisingly, every animal that received sugar in its home cage showed a preference to natural sugar. This preference lasted more than twenty four hours, as the animals continued to demonstrate a clear preference for sugar the following day (FIG. 2).

Taken together with the results of Experiment 1, this result demonstrates that animals form a preference to sugar independent of sweet taste.

### Experiment 3

### Brainstems Neurons Selectively Respond to Sugar

To determine where sugar preference is encoded, applicants screen brain regions for increased expression of Fos, a proxy for neural activity, in animals challenged with sugar but not water or artificial sweetener.

### Experimental Details

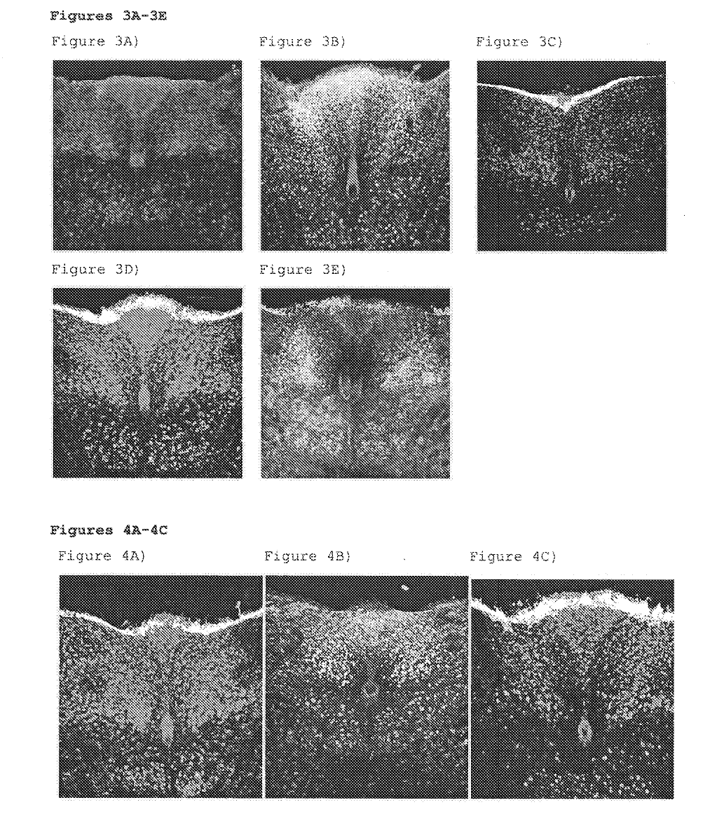

Wild-type animals were water deprived for thirty-six hours and then given access to 1 mL solution of water (FIG. 3A), sucrose (FIG. 3), or artificial sweetener (FIG. 3C). Tasteless (TrpM5 knockout) animals were given water (FIG. 3D) or sucrose (FIG. 3E), The caudal nucleus of the solitary tract was analyzed using cFos staining. Neurons in animals given sucrose are robustly labeled while animals given water or sweetener showed almost no labeled cells at all. An identical pattern of labeling was visible in both wild type and tasteless mice, indicating that the labeling is taste independent. Green: c-fos antibody staining. Magenta: a neural stain (neurotrace) (FIG. 3A-3D).

These experiments revealed a specific increase in the activity of a selective group of neurons in the caudal nucleus of the solitary tract (cNST) of the brainstem, a region known to receive input from the vagus nerve, which conveys information from the viscera (stomach, intestines, etc.) to the brain (FIG. 3). This increase in activity was only in response to sugar and did not occur in animals that ingested artificial sweetener or water, Fos expression patterns were identical in animals unable to taste sweet (T1R2/T1R3 double knockout, data not shown) and those lacking key signal transduction channels (TrpM5; , indicating that the cNST is activated by sugar independent of taste. Taken together, these results identify the cNST as a region of the brain highly activated by the ingestion of sugar, independent of the taste of sweet,

### Experiment 4

### cNST Activity in Responses to Sugar is Triggered by a Post-Oral Mechanism

Because formation of a preference to sugar does not require taste or signaling in the oral cavity, a post-oral mechanism is likely responsible. We hypothesized that infusing a sugar solution directly into the gut should activate cNST neurons in the same manner as animals drinking the same solution.

Wild type animals were infused directly into the gut with 0.5 mL of water (FIG. 4A), sucrose (FIG. 4B), or artificial sweetener (FIG. 4C). Again, neurons in animals given sucrose were robustly labeled while animals given water or sweetener showed almost no labeled cells at all. Green: c-fos antibody staining. Magenta; a neural stain (neurotrace) (FIG. 4A-4C).

As predicted, Fos expression in the cNST is identical when animals gavaged with sucrose, AceK, or water.

### Experiment 5

When given a choice between highly concentrated artificial sweetener and a low concentration of natural sweet, animals consume the sweeter substance. However, after one exposure to natural sugar, animals switch their preference to the natural sweet (See FIG. 5). If the cNST is indeed an essential brain center for establishing sugar preference, a prediction would be that silencing the activity of the neurons in this nucleus during exposure to sugar should prevent a preference from forming. To determine if this is the case, applicants implanted a cannula above the cNST of naive animals, waited 2 weeks for recovery, and then assessed the ability of these animals to form a sugar preference. In initial tests, animals all demonstrated a strong preference for the sweeter artificial compound. Prior to receiving sugar in their home cage, animals were injected with a glutamate receptor antagonist (50 nL of NBQX, 5 pg/mL) to reversibly silence activity in the cNST. Importantly, this silencing does not abolish sweet taste, as the same animals are innately attracted to sweet compounds in short-access assays. Twenty-four hours later, they continued to prefer the artificial sweetener (FIG. 5). As expected, animals regained their ability to form a preference after the drug washed out (FIG. 5).

Taken together, these results demonstrate that the caudal NST is a required neural substrate for the formation of sugar preference.

### Experiment 6

### Sugar-Responsive Brainstem Neurons Encode a Positive Valence

Given that the activity of the cNST is necessary for the formation of a preference to sugar, applicants hypothesized that activation of these neurons should be attractive.

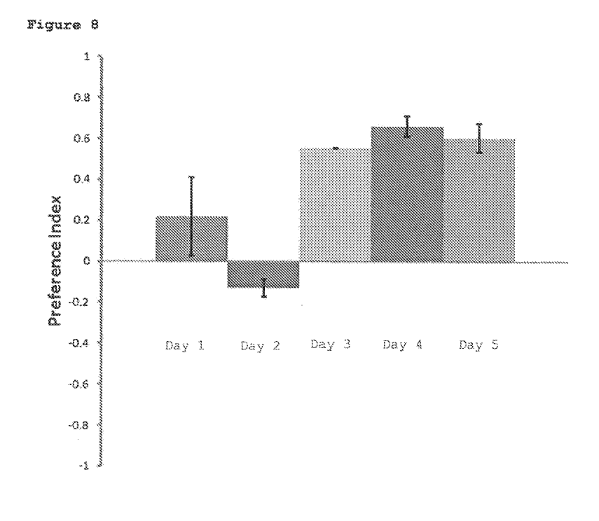

Nine wild type mice were injected with an adeno-associated virus expressing channelrhodopsin-2 under the control of the cFos promoter into the cNST. This system allows exogenous activation of neurons by illuminating them with blue light; furthermore, only neurons that respond to a stimulus will be activated. A fiber was placed over the cNST to allow optical access to the tissue. After allowing the animals to recover for two weeks, applicants challenged each animal with water, sugar, or artificial sweetener. Twelve hours after consuming the solution, each mouse was placed into a two-chamber assay. The presence of the animal in one of the two chambers was coupled to laser-stimulated activity in the sugar-responsive neurons in the cNST. Thus, when the animal enters one chamber, a laser attached to the implanted optical fiber fires, which leads to the activation of channelrhodopsin expressing neurons in the NST. When the animal is in the other chamber, the light is off. The animal's preference for activation of sugar responsive neurons in the cNST was determined as a function of the time spent in the chamber coupled to activation of these neurons versus the chamber without. Animals that consumed sugar show a marked preference for the chamber coupled to activation of the neurons in the NST while animals given artificial sweetener or water do not (FIG. 6).

These results demonstrate that activation of the neurons in the cNST that respond to sugar is highly pleasurable to the animal.

### Experiment 7

### Sugar-Responsive Brainstem Neurons Encode a Positive Valence

Twelve animals were injected with a virus in the cNST expressing Channelrhodopsin under the control of the c-fos promoter and implanted with fiber optics above the site. Two weeks later, six animals were given sugar to induce c-fos driven channelrhodopsin expression. As a control, six animals were given artificial sweetener. Animals were then placed into a chamber with two access ports: one port delivered water and was coupled to laser activation of channelrhodopsin expressing neurons in the NST while the other port delivered water alone. Animals given sugar strongly preferred the port coupled to laser activation while those given artificial sweetener had no preference to either port (FIG. 7).

Our results demonstrate that animals strongly preferred (and will actually self-stimulate) the port coupled to activation of the sugar-responsive cNST neurons.

### Experiment 8

Because neurons in the cNST are necessary for sugar preference formation, applicants reasoned that the activation of these neurons should be sufficient to form a preference to a neutral stimulus. To test this, applicants expressed channelrhodopsin in the sugar responsive cells of the cNST. This time, applicants inject adeno-associated viruses containing a cre-dependent channelrhodopsin gene into the cNST of mice that express Cre-ER under the control of the Arc promoter. Arc is an immediate early gene, similar to Fos, and is frequently used as a proxy for neural activity. These animals therefore express channelrhodopsin in cNST neurons that are highly activated. During the surgery to inject the virus, an optical fiber was also implanted over the cNST to allow optical access to the tissue. The animals were allowed to recover for two weeks. They were then challenged with sugar two hours after being injected with 4-hydroxytamoxifen (50 mg/kg).

Two weeks later, the animals were tested for their preference between water and a low concentration of sodium chloride, which mice can taste but have no innate preference for or against. Animals clearly showed no bias for either the water or the salt (FIG. 12). The next day, animals were given access to the same salt solution in the home cage. When the animals drank, a touch detector sensed each lick and triggered a laser to illuminate the cNST with blue light, triggering activity of the neurons in the cNST. Twenty tour hours later, animals were again tested for a preference between water and the salt solution. Animals clearly demonstrate a preference for the previously neutral salt solution after they had learned to associate activation of sugar-responsive cNST neurons with the taste of the salt solution (FIG. 8). These results clearly show that activation of sugar responsive cells in the cNST is capable of forming a preference to a neutral stimulus.

### Experiment 9

A satiated animal was given 32 mM sucralose for the first 11 trials, and then given either 32 mM sucralose alone, or 32 mM sucralose+0.4 M MDG. Note that the presence of MDG dramatically increases the appetitiveness/consumption of sucralose (FIG. 9). Shown are 20 trials.

### REFERENCES

Yang, Qing. “Gain weight by “going diet?” Artificial sweeteners and the neurobiology of sugar cravings: Neuroscience 2010. ”The Yale journal of biology and medicine 83, no. 2 (2010): 101.

What is claimed is:

1. A method of modulating the craving and/or desire for natural sugar in a subject, comprising agonizing or stimulating a selective group of neurons in the caudal nucleus of the solitary tract (cNST) of the brain in the subject, either directly or via the gut or gut-brain axis.

2. A method of modulating the craving and/or desire for natural sugar in a subject, comprising antagonizing or silencing a selective group of neurons in the caudal nucleus of the solitary tract (cNST) of the brain in the subject, either directly or via the gut or gut-brain axis.

3. The method of claims 1 or 2, wherein the neurons are agonized or stimulated or antagonized or silenced by the administration of a pharmaceutical composition to the subject.

4. The method of claim 2, wherein the neurons are antagonized or silenced by the administration of a neural, silencer to the subject.

5. The method of claim 4, wherein the neural silencer is a glutamate receptor antagonist.

6. The method of claim 4, wherein the neural silencer is NBQX.

7. The method of claims 1 or 2, wherein the neurons are agonized or stimulated or antagonized or silenced before or during ingestion of a natural sugar or a food or beverage product containing natural sugar.

8. The method of claim 3, wherein the pharmaceutical composition is administered before or during the ingestion of a natural sugar or a food or beverage product containing natural sugar.

9. The method of claim 4, wherein the neural silencer is administered before or during the ingestion of a natural sugar or a food or beverage product containing natural sugar.

10. A composition for modulating the craving and/or desire for natural sugar, wherein the composition antagonizes or silences a selective group of neurons in the caudal nucleus of the solitary tract (cNST) of the brain in the subject, either directly or via the gut or gut-brain axis.

11. A food or beverage product comprising natural sugar and the composition of claim 10.

12. A composition for modulating the craving and/or desire for natural sugar, wherein the composition agonizes or stimulates a selective group of neurons in the caudal nucleus of the solitary tract (cNST) of the brain in the subject, either directly or via the gut or gut-brain axis.

13. A food or beverage product comprising natural sugar and the composition of claim 12.

14. The methods of claims 1-1 wherein the subject is a mammal.

15. The method of claim 14, wherein the subject is a mouse.

16. The method of claim 14, wherein the subject, is a human.

17. The compositions of claims 10 and 12, wherein the subject is a mammal.

18. The composition of claim 17, wherein the subject is human.

19. A method for identifying a composition or agent for modulating the craving or desire for natural sugar, comprising:
a) administering a natural sugar to a mouse;
b) detecting neural activity in the neurons in the caudal nucleus of the solitary tract (cNST) of the brain;
c) administering the composition or agent to the mouse;
d) detecting neural activity in the neurons in the caudal nucleus of the solitary tract (cNST) of the brain;
e) comparing the activity in step (d) with the activity in step (b),
wherein a decrease or less activity in step (d) as compared to step (b) indicates that the agent or composition is decreasing the craving or desire for natural sugar, and an increase or more activity in step (d) as compared to step (b) indicates the agent or composition is increasing the craving or desire for natural sugar.

20. The method of claim 19 wherein detecting neural activity in the neurons in the caudal nucleus of the solitary tract (cNST) of the brain is accomplished by staining the brain of the mouse.

21. A method of increasing an individual's preference for a consumer product which comprises adding to said consumer product a non-metabolizable sugar analog capable of activating a gut-brain sweet preference circuit in an amount effective to activate such circuit.

22. A method of maintaining an individual's preference for a consumer product while reducing, its metabolizable sugar content which comprises adding to said consumer product a non-metabolizable, sugar analog capable of activating a gut-brain sweet preference circuit in an amount effective to activate such circuit.

23. The method of claim 21 or 22, wherein the non-metabolizable sugar analog is further capable of activating the sweet taste receptors on the individual's tongue.

24. The method of claim 21 or 22, which further comprises adding to said consumer product an artificial sweetener, a sugar substitute, or a compound which activates the sweet taste receptors on the individual's tongue.

25. The method of any one of claims 21-24, wherein the non-metabolizable sugar analog is selected from Alpha-Methyl-D-Glucopyranose, Beta-D-Glucose, D-Allopyranose, Beta-L-fucose, Alpha-D-Fucose, 6-Deoxy-Alpha-D-Glucose, Beta-D-Fucose, 6-Deoxyglucose, Alpha-L-Fucose, Ribose, Alpha-L-Arabinose, Beta-L-Arabinose, Galacturonic Acid, D-Mannuronic Acid, L-Iduronic Acid, D-Glucuronic Acid, L-Glucuronic Acid, L-Glycero-D-Manno-Heptopyranose, Alpha-D-Xylopyranose, L-Xylopyranose, Beta-D-Ribopyranose 2-O-Methyl Fucose, 6-Deoxy-2-O-Methyl-Alpha-L-Galactopyranose, Methyl Alpha-D-mannoside, Methyl Alpha-galactoside, Methyl Beta-galactoside, Alpha-D-Glucose-6-Phosphate, Beta-Galactose-6-Phosphate, Alpha-D-Mannose-6-Phosphate, Beta-D-Glucose-6-Phosphate, 3,4-Epoxybutyl-Alpha-D-Glucopyranoside, 2-Deoxy-Beta-D-Galactose, 2-deoxyglucose, D-Galctopyranosyl-1-On, Gluconolactone, 1-Thio-Beta-D-Glucopyranose, O1-Pentyl-Mannose, 5 (R)-5-Fluoro-Beta-D-Xylopyranosyl-Enzyme Intermediate, D-Sorbitol, Mannitol, D-Xylitol, Beta-L-Methyl-Fucose, Alpha-L-Methyl-Fucose, Alpha-L-1-Methyl-Fucose, L-Rhamnitol, Fucitol, O3-Sulfonylgalactose, O4-Sulfonylgalactose, Gluconic Acid, Methyl(6s)-1-Thio-L-Manno-Hexodialdo-6,2-Pyranoside, 1-N-Acetyl-Beta-D-Glucosamine, Alpha-D-Glucopyranosyl-2-Carboxylic Acid Amide, D-Glucose in Linear Form, 02-Sulfo-Glucuronic Acid, 4-O-Methyl-Beta-D-Glucuronic Acid, 4-O-Methyl-Alpha-D-Glucuronic Acid, 1-Deoxy-1-Methoxycarbamido-Beta-D-Glucopyranose, Alpha-D-Galactose-1-Phosphate, D-Mannose 1-Phosphate, Alpha-D-Glucose-1-Phosphate, 1-(Isopropylthio)-Beta-Galactopyranside, 2-(Beta-D-Glucopyranosyl)-5-Methyl-1, 3, 4-Oxadiazole, 5-(3-Amino-4, 4-Dihyroxy-Butylsulfanylmethyl)-Tetrahydro-Furan-2,3,4-Triol, Beta-D-Arabinofuranose-5′-Phosphate, [(2r,3s,4s,5r)-3,4,5-Trihydroxytetrahydrofuran-2-Yl ]Methyl Dihydrogen Phosphate, L-Rhamnose, Myo-Inositol, Glucarate, 3,6-Anhydro-D-Galactose-2-Sulfate, 4-Deoxy-Alpha-D-Glucose, Tetrahydrooxazine, D-Fructose-6-Phosphate, Sorbitol 6-phosphate, 2-Deoxy-Glucose-6-Phosphate, 2-Deoxy-2-Aminogalactose, Glucosamine, 2-Fluoro-2-Deoxy-Beta-D-Galactopyranose, 2-Deoxy-2-Fluoro-Alpha-D-Mannose, 2-Deoxy-2fluoro-Glucose, 2-Deoxy-2-Fluoro-Beta-D-Mannose, L-Guluronic Acid 6-Phosphate, 6-Phosphogluconic Acid, L-Myo-Inositol-1-Phosphate, 4, 6-Dideoxyglucose, 2-Deoxy-2-Fluoro-Alpha-D-Mannosyl Fluoride, 4-Deoxy-D-Glucuronic Acid, Fructose, Glucose-6-Phosphate, Beta-D-Fructopyranose, 1-Deoxy-Ribofuranose-5′-Phosphate, Tagatose, Ribose-1- Phosphate, Fructose-6- Phosphate, 5-Hydroxymethyl-Chonduritol, 3-Deoxy-D-Manno-Oct-2-Ulosonic Acid, 2-Deoxy-D-Glucitol 6-(E)-Vinylhomophosphonate, D-Treitol, Meso-Erythritol, Xylarohydroxamate, or C-(1 -Hydrogyl-Beta-D-Glucopyranosyl) Formamide.

26. The method of claim 25, wherein the non-metabolizable sugar analog is Alpha-Methyl-D-Glucopyranose.
